(19) United States
(12) Patent Application Publication
(10) Pub. No.: US 2004/0197787 A1
(43) Pub. Date: Oct. 7, 2004

(54) Affinity reaction probe detection/analysis chips and detection system and apparatus using the same

(76) Inventor: Hiroshi Nagasawa, Osaka (JP)

Correspondence Address: BROWDY AND NEIMARK, P.L.L.C., 624 NINTH STREET, NW, SUITE 300, WASHINGTON, DC, 20001-5303, US

(21) Appl. No.: 10/477,146

(22) PCT Filed: May 9, 2002
(86) PCT No.: PCT/JP02/04531

(30) Foreign Application Priority Data
May 9, 2001 (JP) 2001-138855

(51) Int. Cl. C12Q 1/68; C12M 1/34

(52) U.S. Cl. 435/6; 435/287.2

(57) ABSTRACT

Affinity reaction probe detection/analysis chips each comprising a retainer ring that is marked with individual identifying signals and which has a through-hole filled with or retaining a carrier having probe molecules fixed thereto. The carrier having probe molecules fixed thereto is preferably a porous membrane or nonwoven fabric stretched across the carrier to close the hole. The individual identifying signals may be written according to either a barcode system or an electronic recognition system.

To establish a more convenient detection system in a chip-based affinity reaction probe assay, reaction detection/analysis chips are provided that can be used to detect DNA polymorph and diagnose various physiological functions. A detection system using such chips is also provided.

### TECHNICAL FIELD

This invention relates to affinity detection/analysis chips that enable a number of functional molecules to be recognized as they are used typically in genetic diagnosis and diagnosis of physiological functions. The invention also relates to a process for preparing such chips, as well as a detection system and a detection apparatus using such chips.

### BACKGROUND ART

In affinity detection, a substance that binds selectively to a specific molecule is used to achieve selective detection of a corresponding substance; this is a very sensitive detection method and has typically been used in liquid chromatography as an affinity column to detect a specific protein using a specific enzyme. However, the affinity approach used in liquid chromatography only gives information about the specific molecule and it is not a means of analysis that can handle a number of molecules and which permits simultaneous supply of information about their existence.

Currently, detection of polymorph due, for example, to gene variations, particularly those in a single base (sequence) is not only effective in diagnosis of mutation or otherwise induced diseases such as cancer but also necessary to provide a guideline for evaluating drug response and side effects, as well as contributing to analysis of and predictive medicine on etiologically related genes for multi-factorial diseases. It is known that the use of so-called DNA chips as a type of the affinity process is effective in detection for these purposes.

Conventionally used DNA chips with short fixed DNA chains which are available from Affymetrix under the name “GeneChip” are typically silicon or glass substrates about 1 cm square, each having more than ten thousand oligo-DNA fragments (DNA probes) fabricated on their surfaces by photolithographic techniques. When a DNA sample to be assayed, which is typically fluorescence labelled, is flowed onto the DNA chip, a DNA fragment having a complementary sequence to one of the probes on the chip binds to said probe and the linkage can be identified by fluorescence so that the specific sequences of DNA fragments in the DNA sample are recognized and quantitated. This method has been shown to be capable of detecting mutations in cancer genes and genetic polymorph.

Also used today are microarrays having a plurality of cDNAs arranged on slide glass.

### DISCLOSURE OF INVENTION

These chip-based affinity reaction probe methods are very effective as means of analysis by which the information about existence can be offered simultaneously for a number of molecules. On the other hand, they have several problems. For example, to synthesize one stage of DNA chip using photolithography, at least four photomasks are necessary and a process consisting of photolithographic, coupling and cleaning steps must be performed four times. Since these procedures must be repeated as many times as are required to produce the desired chain length, the synthesis cost is inevitably increased. In addition, the photomask need be changed for each varying pattern, making it impossible to achieve flexible preparation of DNA chips of various designs according to specific needs. What is more, single-stage synthesis does not guarantee the required quality of individual spots and the problem of persistent instability in chip-to-chip reproduction remains unsolved.

The DNA chips of a microarray type have been proposed as an alternative to this approach. Being prepared by high-density spotting of a synthesized oligonucleotide solution, this type of chips permit easier pattern changes than in the method using lithography. On the other hand, probe molecules have to be spotted point by point on a fixing glass plate and the like, rendering the synthesis cost as high as in the case of using photolithography to prepare DNA chips.

A further problem with either type of reaction chips is that during detection, hybridization is localized to defy quantification and hybridization reactions need be carried out for a prolonged time using a dedicated apparatus. Under the circumstances, detecting various kinds of genetic information as in bone marrow transplantation requires not only much labor and time but also considerable expenses.

An object, therefore, of the present invention is to provide a reaction detecting system that is more convenient to use and which still can be used to diagnose DNA polymorph and various other physiological functions.

In order to meet this object, the present inventors made various studies for establishment of a convenient detection system without using the photolithographic techniques that involve lengthy reaction steps, that are complicated, that present difficulty in adapting themselves flexible enough to different objectives and that are very costly, and without using a dedicated hybridizing apparatus.

The inventors found that the foregoing problems with the prior art could be solved by a detection system that was constructed by stacking a number of detection chips capable of undergoing single individual reactions and being labelled with individual identifying signals in order to enable verification of the probe molecule entering that has entered into a reaction. The present invention has been accomplished on the basis of this finding.

### BRIEF DESCRIPTION OF THE DRAWINGS

FIG. 1A is a plan view of a retainer ring having a through-hole in which is retained a reactive probe fixing carrier;

FIG. 1B is a cross section of FIG. 1A;

FIGS. 2A-2D show in cross section four examples of the reaction field within the through-hole in the retainer ring;

FIG. 3A is a plan view of a retainer ring provided on a surface with annular bar codes as identification marks;

FIG. 3B is a plan view of a retainer ring provided on a surface with dot matrix bar codes as identification marks;

FIG. 4 is a sketch showing how probe molecules such as cDNA are fixed to a surface of the carrier using a linker;

FIG. 5 is a sketch showing how oligonucleotides synthesized as probe molecules are fixed to a surface of the carrier using a linker;

FIG. 6 is a sketch showing how synthesis reagents or probe molecules are successively flowed through a stack of carrier retaining chips so that the probe molecules are fixed to the chips;

FIG. 7 is a sketch showing how a reaction probe chip assembly, or a stack of chips that have probe molecules fixed for undergoing respectively different specific binding reactions, is supplied with a flowing sample solution for detection;

FIG. 8 shows how chips in a holder that have passed through all reactions involved are loaded one by one in a fluorescence detector;

FIG. 9 shows in conceptual form a probe molecule fixing and preparing apparatus in which chips in a stack are successively supplied with either a flow of reaction reagents by means of a reagent supplier to synthesize oligonucleotides via an on-off valve and a pump or a flow of cDNA to be fixed;

FIG. 10 shows in conceptual form an apparatus for use with a light absorbing detection system that recovers the chips one by one as they have passed through the intended reactions and which reads the identification marks on the chips; and

FIG. 11 is a diagrammatic representation of a hybridizing apparatus and a fluorescence observing apparatus.

### DESCRIPTION OF THE INVENTION

The present invention successfully solved the prior art problems by either one of the means set forth below. (1) An affinity reaction probe detection/analysis chip comprising a retainer ring that is labelled with individual identifying signals and which has a through-hole filled with or retaining a carrier having probe molecules fixed thereto. (2) A process for preparing affinity reaction probe detection/analysis chips, comprising the steps of stacking chips each comprising a retainer ring that is labelled with individual identifying signals and which has a through-hole filled with or retaining a carrier, passing a solution of preliminarily synthesized or naturally extracted probe molecules through said through-hole, and fixing said probe molecules to a surface of said carrier using a linker.

(3) A process for preparing affinity reaction probe detection/analysis chips, comprising the steps of stacking chips each comprising a retainer ring that is labelled with individual identifying signals and which has a through-hole filled with or retaining a carrier, and synthesizing oligonucleotides as probe molecules on a surface of said carrier.

(4) An affinity reaction probe detection/analysis chip assembly which is a stack of the affinity reaction probe detection/analysis chips according to (1) above, wherein the probe molecules fixed to inner surfaces of the carrier are capable of causing different specific binding reactions, said through-holes being joined to form a channel through which an analyte can pass.

(5) An apparatus for preparing affinity reaction probe detection/analysis chips, in which chips each comprising a retainer ring that is labelled with individual identifying signals and which has a through-hole filled with or retaining a carrier are stacked and reagents are successively flowed through said through-holes as stated in (2) or (3) above.

### EMBODIMENTS OF THE INVENTION

Modes for carrying out the invention are described below in detail with reference to the accompanying drawings.

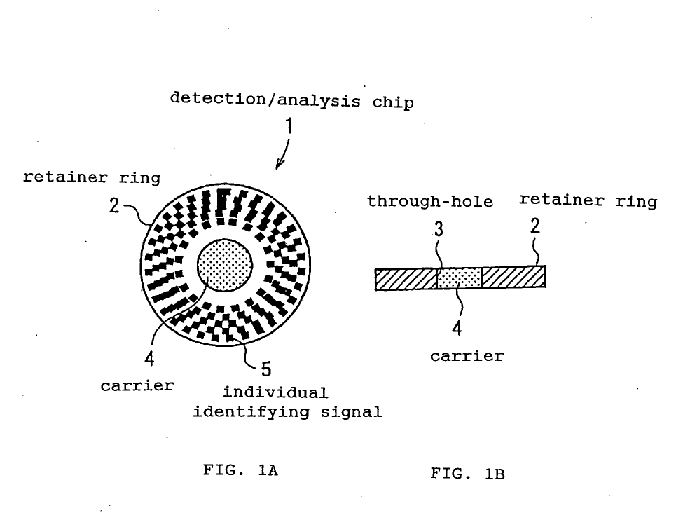

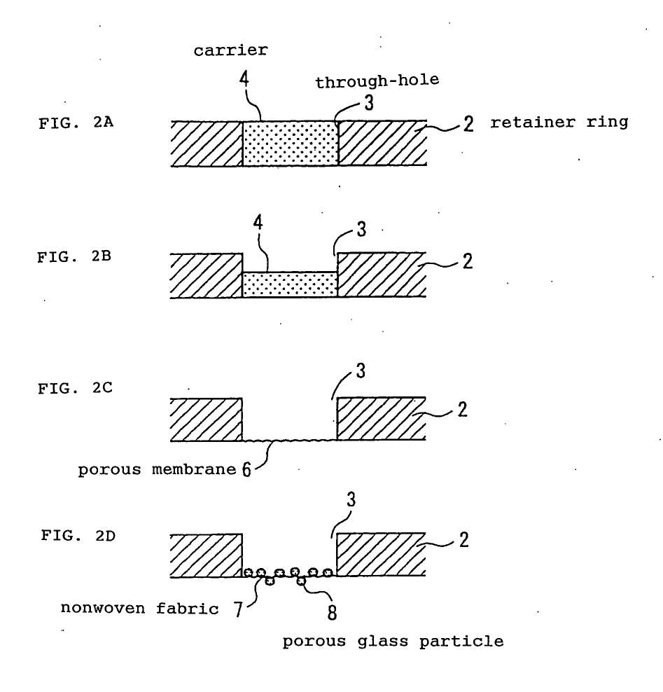

FIG. 1A is a plan view of a retainer ring 2 having a through-hole 3 in which is retained a reactive probe fixing carrier 4 and FIG. 1B is a cross section of FIG. 1A. The reaction site within the through-hole may take various forms as shown in FIGS. 2A-2D. The only requirement is that the through-hole be large enough to permit passage of an analyte and be capable of retaining reactive probe molecules.

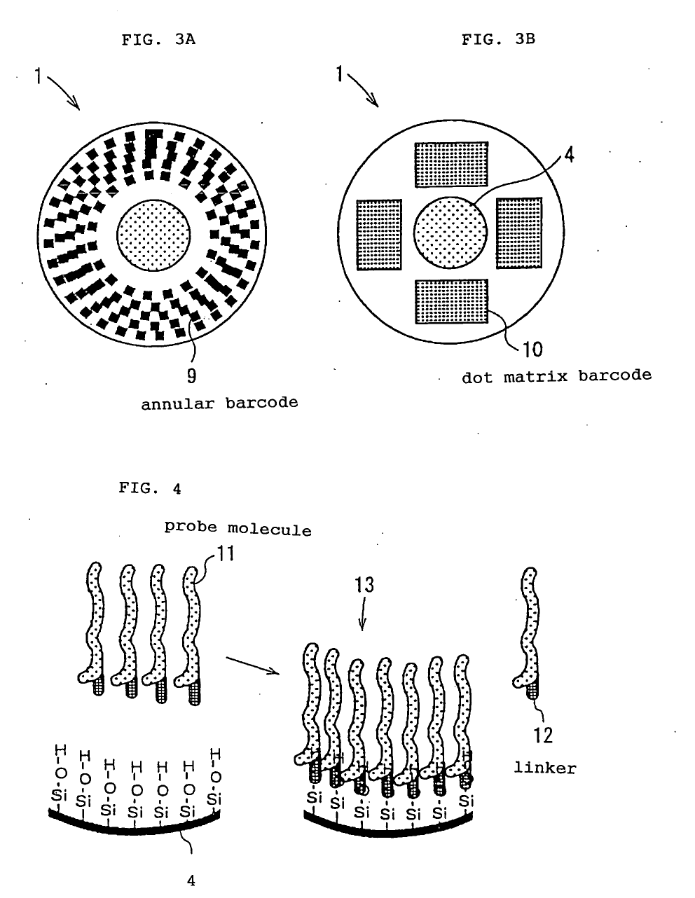

FIG. 3A shows a detection/analysis chip 1 provided on a surface with annular bar codes 9 as identification marks 5 and FIG. 3B is a plan view of a detection/analysis chip 1 provided on a surface with dot matrix bar codes 10 as identification marks 5.

FIG. 4 shows how probe molecules 11 such as cDNA are fixed to a surface of the carrier 4 using a linker 12.

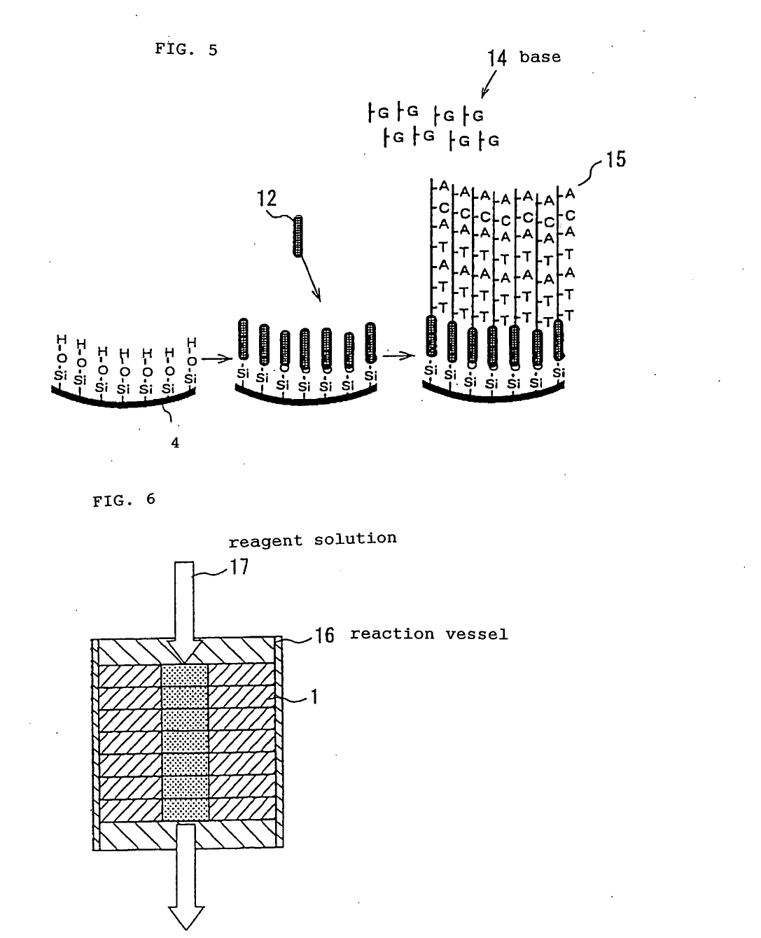

FIG. 5 shows how oligonucleotides 15 synthesized as probe molecules from bases 14 are fixed to a surface of the carrier 4 using a linker 12.

FIG. 6 shows how synthesis reagents 17 or probe molecules are successively flowed through a stack of carrier retaining chips 1 in a reaction vessel 16 so that the probe molecules are fixed to the chips, whereby a multiple of identical chips can be prepared simultaneously.

FIG. 7 shows how a reaction probe chip assembly 20, or a stack of chips that have probe molecules fixed to inner surfaces for undergoing respectively different specific binding reactions, in a holder 18 is supplied with a flowing sample solution 19 for detection.

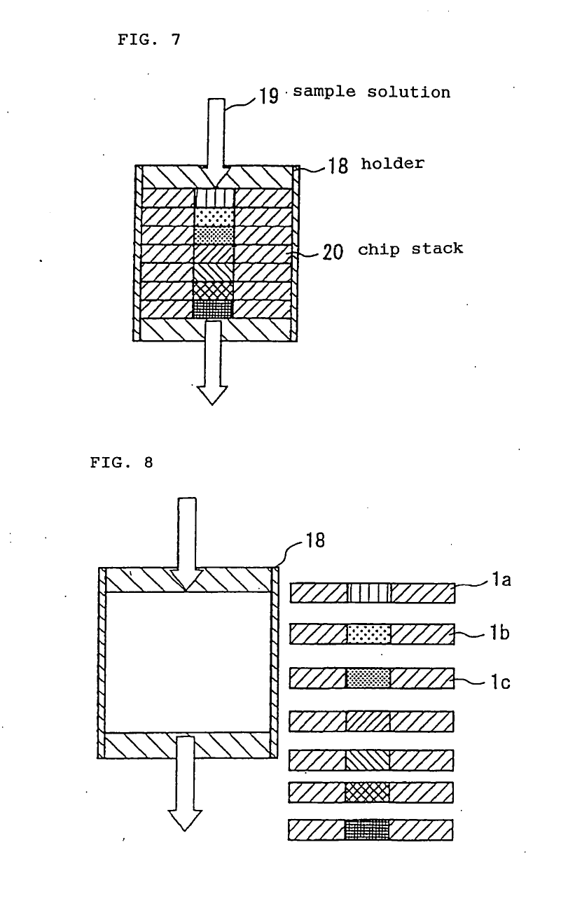

FIG. 8 shows a detection method in which chips 1a, 1b, 1c, . . . fixed in the holder 18 that have passed through all reactions involved are taken out one by one for loading in a fluorescence detector.

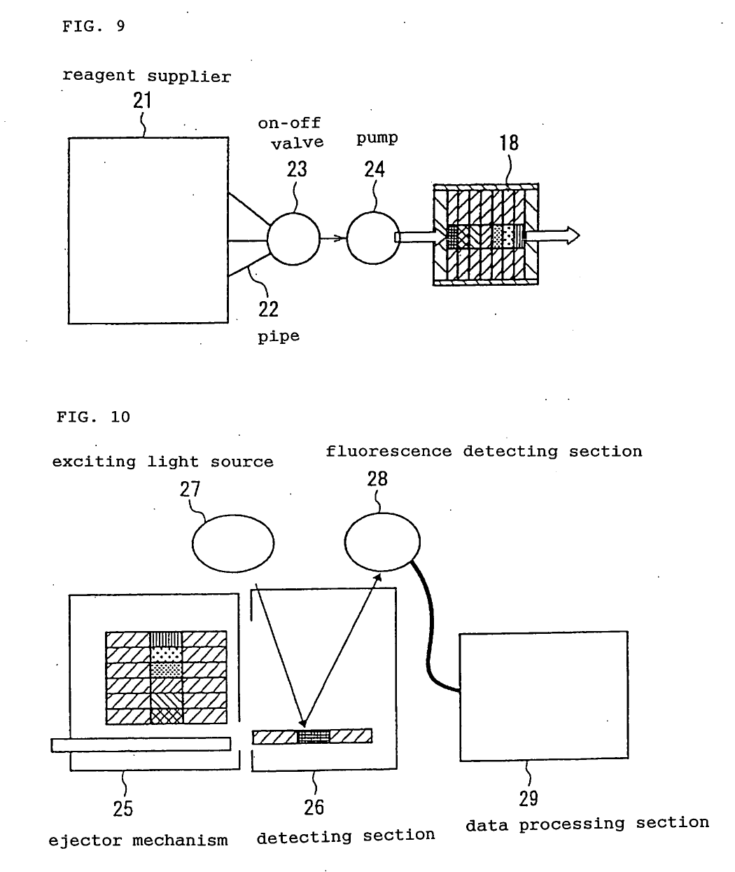

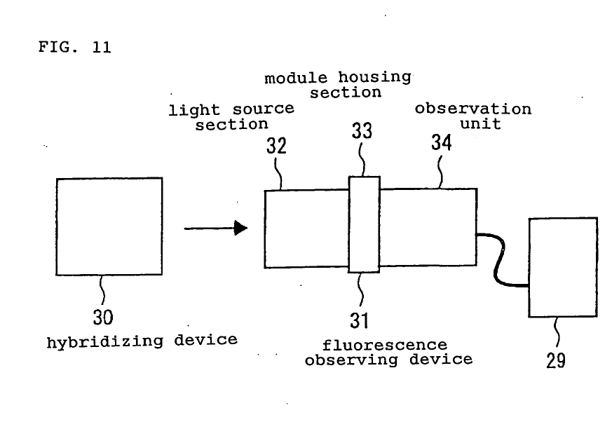

FIG. 9 shows in conceptual form a probe molecule fixing and preparing apparatus in which chips in a stack are successively supplied with a flow of reaction reagents from a reagent supplier 21 through pipes 22, an on-off valve 23 and a pump 24 to synthesize oligonucleotides or supplied with a flow of cDNA to be fixed.

FIG. 10 shows in conceptual form an apparatus for use with a light absorption detecting system that reads the chips as they have passed through the intended reactions. The chips are taken out one by one from an ejector mechanism 25; an exciting light source 27 is applied to a detecting section 26 and the emitted fluorescence is detected by a fluorescence detecting section 28 so that the identification signals are read as the individual chips are successively fed into the detecting section 26; the identification signals thus read are processed for detection in a data processing section 29.

FIG. 11 is a diagrammatic representation of FIG. 10. The product of hybridization with a hybridizing apparatus 30 is supplied into a fluorescence observing apparatus 31 comprising a light source unit 32, a module housing unit 33 and an observation unit 34, where it is subjected to fluorescence detection and finally detected by the data processor 29. The reaction probe assembly in principle comprises a number of reaction probe chips but depending on the case it may consist of only one chip.

We next describe the constituent material, size and other design factors of the carrier and other components shown in the accompanying drawings.

Examples of the probe that can be used in the affinity reaction detection/analysis chip of the invention include DNA, RNA or PNA (peptide nucleic acid) or fragments thereof, oligonucleotides having any desired base sequences, antigens, antibodies, epitopes, enzymes, proteins and polypeptide chains at their functional sites. It should, however, be noted that any other materials useful as probes can be used in the invention.

Preferred examples of the carrier material for supporting the probes mentioned above include porous materials such as the powder of porous glass (indicated by 8 in FIG. 2D), porous membranes (indicated by 6 in FIG. 2C) and nonwoven fabrics (indicated by 7 in FIG. 2D). However, the holes in these porous materials may be of any shape as long as they have the porous structure. To be more specific, the porous materials providing the reaction field may be of any type that is capable of fixing the molecules of the affinity reaction probe or causing them to grow and particularly preferred examples are porous glass and filter paper made of glass fibers.

The pore size of the porous glass powder and membranes is preferably between 0.1 and 0.5 μm whereas the fine pores in nonwoven fabrics and filter paper are preferably not greater than a few μm. Too small pores render the filtering of fluorescence labelled samples difficult, so pores of at least 0.1 μm are required.

The retainer ring has a center through-hole in which is retained the carrier having probe molecules fixed thereto. While the retainer ring has several features, one of them is that each ring has identification signals marked on a surface to describe its characteristics (e.g. the number and type of probes and the structure and constituent material of the carrier).

The diameter of the retainer ring ranges preferably from 3 mm to 10 mm, more preferably from 5 mm to 7 mm, and its thickness ranges preferably from 0.1 mm to 1 mm, more preferably from 0.1 mm to 0.5 mm.

The retainer ring may be formed of any materials that are stable in shape, have necessary physical strength and are less likely to deteriorate due to dissolution and corrosion. Preferred examples of such materials are resins including poly(ether ether ketone) (PEEK), polypropylene (PP) and poly(ethylene terephthalate) (PET). Also useful are glass, ceramics and metals such as aluminum. Particularly preferred are borosilicate glass, quartz glass and PEEK. The retainer ring may have any shapes as long as its function is exhibited; it may be an ordinary ring which is a disk having a circular center through-hole as shown in FIG. 1; alternatively, it may be a square card having a circular or square center through-hole.

The through-hole in the retainer ring are generally from 0.1 μm to 5 mm, preferably 0.5 μm to 3 mm. However, the through-hole need only be large enough to permit the passage of genes and other analytes; preferably, it may range from 0.1 μm to 10 μm, more preferably from 0.5 μm to 5 μm.

The individual identifying information may be of any form selected from annular or dot matrix bar code systems and electronic recognition systems such as IC tags. The only requirement is that such information be written on the retainer ring. While either color-based signals or embossed signals will do, signal forms that do not interfere with the stacking mechanism are used in practice. There are no particular limitations on the signal form if it is not necessary to stack retainer rings.

On the pages that follow, the structure of the affinity reaction probe detection/analysis chip of the invention, the method of its fabrication, the reaction involved, the procedure of detection, and the relevant system and apparatus are described in detail.

(Structure)

The affinity reaction probe detection/analysis chip of the invention which is indicated by 1 in FIG. 1 comprises a retainer ring 2 in the form of a circular substrate which has a center through-hole 3 in which is filled or otherwise retained in a carrier 4 having a reactive probe fixed thereto. In the reaction field where the carrier 4 exists, probe molecules are fixed as shown by 11 in FIG. 4 and individual identifying signals 5 are written on the retainer ring 2. The carrier 4 may be in the form of a porous membrane 6 (see FIG. 2C) or a nonwoven fabric 7 (FIG. 2D).

The reaction field within the through-hole 3 may assume various forms as shown in FIG. 2. Whichever form is used, it suffices for the through-hole 3 to have a sufficient size to permit the passage of genes and other analytes while retaining probe molecules capable of reaction. In FIG. 2A, the through-hole 3 is completely filled with the carrier 4; in FIG. 2B, the through-hole 3 is filled with the carrier 4 to about one half its depth; in FIG. 2C, a porous membrane 6 is stretched across the bottom of the through-hole 3; in FIG. 2D, a nonwoven fabric 7 is likewise stretched across the bottom of the through-hole 3, with porous glass particles adhering to the upper and lower surfaces of the nonwoven fabric 7. The porous glass particles are preferably of such a structure that they are intertwined with or coupled to the fibers in the nonwoven fabric 7.

(Method of Fabrication)

A plurality of chips 1 having the design described above are stacked as shown in FIG. 6 and a solution of reaction reagents is applied continuously so that it flows through successive through-holes 3, whereupon oligonucleotides are synthesized on each carrier by a suitable method such as the phosphamidite process; the synthesized oligonucleotides may be used as affinity reaction probe molecules. As shown in FIG. 6, synthesis within each of the stacked through-holes can simultaneously yield a multiple of chips having identical characteristics. No problem occurs even if the chips are not stacked but more uniform reaction occurs to yield chips of better characteristics if the chips are stacked as shown in FIG. 6.

If cDNA is flowed through the holes in stacked chips, multiple layers of cDNA can be supported simultaneously on the respective carriers, thus allowing cDNA fixing chips to be prepared with greater ease (FIG. 6). Whichever method is used, the product chips have identical characteristics which can be easily verified by sampling tests; this offers an advantage in quality control.

(Reactions Involved)

For detection, the prepared chips are stacked to form an assembly as indicated by 20 in FIG. 7. In this case, different affinity reaction probe detection/analysis chips are combined to meet a specific need and are preferably fixed within a suitable holder (vessel) 18. If the intended reaction is expected to proceed adequately, the chips need not always be stacked but may be arranged to be capable of free movement on the condition that their identification signals will not be interfered with.

After the chips are assembled in a stack, a sample such as fluorescently labelled cDNA passes through the through-holes in the chips either by suction or under pressure. As a result, the sample can pass by the probe molecules to permit easy hybridization. Hence, the reaction proceeds rapidly and uniformly to permit the use of a simplified hybridization apparatus (FIG. 7).

(Detection)

After the reaction ends, the chips are loaded one by one into a fluorescence detector 26 as the identification signals are read simultaneously. The chips that have been read are usually discarded but, depending on the case, they may be stored and subjected to extraction to recover a specific DNA.

(Detection System and Apparatus)

In order to prepare the detection/analysis chips described above, a dedicated reactor is necessary. FIG. 9 shows in conceptual form an apparatus in which reaction reagents are successively flowed through stacked chips to synthesize oligonucleotides or, alternatively, cDNA or the like is flowed and fixed.

For detection, a certain type of light absorption detecting system is necessary that can read the identification signals on the chips that underwent the intended reactions. FIG. 10 shows in conceptual form an apparatus that can be used to implement the system; the apparatus allows feeding of the individual chips and reads their identification signals to perform detection. A flowsheet for hybridization and detection procedures is shown in FIG. 11.

### EXAMPLES

The following examples are provided for further illustrating the present invention but are in no way intended to limit its scope.

### Example 1

A number of retainer rings were provided; they were annular poly(ethylene terephthalate) substrates each having a central through-hole of 2 mm^{φ} and measuring 0.3 mm thick by 5 mm across. The through-holes were bored in exact positions such that they would be in registry with each other when the retainer rings are stacked. Filter paper made of glass fibers was placed between adjacent retainer rings and fused to prepare reaction chips. As a result of this process, each reaction chip had a thickness of 0.6 mm. Bar codes of a dot matrix type were printed on the surface of each ring. One hundred of such retainer rings were stacked and reagents were successively passed through the holes in these rings to synthesize oligonucleotides of specified structures. Fifty of the thus prepared individual reaction chips were assembled to form a stack; these chips had oligonucleotides of different structures and were marked with their identification signals.

The stack of reaction chips was placed into a polypropylene reaction cell and subjected to reaction with fluorescence labelled cDNA under analysis. After the reaction, the chips were cleaned, taken out of the cell and analyzed with a fluorescence detector one by one while at the same time their identity was recorded.

### Example 2

A number of retainer rings were provided; they were quartz glass substrates each having a center through-hole of 1 mm^{φ}. The through-holes were bored in as exact positions as in Example 1. They were filled with porous glass particles of 10 μm^{φ} (pore size=100 nm) which were then sintered. The top and bottom surfaces of each retainer ring were polished smooth. Identification signals, either annular or in dot matrices, were written onto the substrate by etching. The retainer rings were chemically cleaned and had their surface aminated with a silane coupling agent. One hundred of such retainer rings were stacked and different kinds of cDNA were fixed in the through-holes by a conventional method.

The stack of retainer rings were put into a polypropylene reaction cell, through which cDNA as a fluorescence labelled analyte was flowed to effect reaction. After the reaction, the chips were cleaned, taken out of the cell and analyzed with a fluorescence detector.

### Example 3

A number of retainer rings were provided; they were PEEK substrates each measuring 10 mm across and 1 mm thick and having a through-hole of 0.5 mm^{φ}. A porous membrane made of regenerated cellulose was stretched across the top surface of each retainer ring. In a separate step, porous glass particles of 5 μm^{φ} (pore size=100 nm) were provided and fixed on the surfaces of the porous membrane using a regenerated cellulose (viscose) solution. Five hundred of such retainer rings were stacked and a solution of reaction reagents was passed through the stack of retainer rings from the bottom, thereby synthesizing various kinds of oligonucleotide. The stack of retainer rings was put into a polypropylene reaction cell, through which cDNA as a fluorescence labelled analyte was flowed to effect reaction. After the reaction, the chips were cleaned, taken out of the cell and individually analyzed with a fluorescence detector.

A number of retainer rings are provided; they are PEEK substrates each measuring 5 mm across and 0.3 mm thick and having a through-hole of 10 μm^{φ}. A porous membrane made of regenerated cellulose is stretched across the top surface of each retainer ring. In a separate step, porous glass particles of 3 μm^{φ} (pore size=100 nm) are provided and fixed on the surfaces of the porous membrane using a regenerated cellulose (viscose) solution. Fifty of such retainer rings are stacked and a solution of reaction reagents is passed through the stack of retainer rings from the bottom, thereby synthesizing various kinds of oligonucleotide. The stack of retainer rings is put into a polypropylene reaction cell, through which cDNA as a fluorescence labelled analyte is flowed to effect reaction. After the reaction, the chips are cleaned, taken out of the cell and individually analyzed with a fluorescence detector.

### Example 5

Example 4 is repeated except for using retainer rings which are PEEK substrates each measuring 5 mm across and 0.3 mm thick and having a through-hole of 5 μm^{φ}. Fifty of such retainer rings are treated as in Example 4 to obtain the desired chips.

Industrial Applicability

According to the invention, there is easily provided a means of analysis which does not require any special equipment such as photolithographic systems but which uses reactive probes such as proteins of any desired structures or oligonucleotides having any desired base sequences that bind selectively to specified molecules to enable selective detection of corresponding substances; this means of analysis is also capable of simultaneously providing information about the existence of many molecules while serving as a detection means capable of high-sensitivity analysis without undue burden.

If unit reaction chips supporting various kinds of reactive substances are provided in advance, affinity reaction probe detection/analysis chip sets that have various kinds of reactive probes fixed and which are guaranteed for reactivity can be supplied as required, in a more convenient way and in the required combinations.

The invention can further provide a reaction probe detection/analysis chip system at lower cost with higher stability.

Hence, in accordance with specific needs of individual persons, suitable reaction probe detection/analysis chip systems for DNA and other analytes can be constructed, thus contributing to custom-made medical practices.

As a further advantage, the affinity reaction probe detection/analysis chip of the invention permits easier control of reaction conditions such as temperature compared to the conventional “DNA chips” and hence offers the opportunity to provide a detection means in new areas such as protein detection.

1. An affinity reaction probe detection/analysis chip comprising:
a retainer ring that is labelled with individual identifying signals and which has a through-hole in it; and a carrier filled or retained in the through-hole and which has probe molecules fixed thereto.

2. The analysis chip according to claim 1, wherein the carrier having said probe molecules fixed thereto is a material having a porous structure.

3. The analysis chip according to claim 2, wherein said carrier is porous glass particles, a porous glass membrane, a nonwoven fabric of glass fibers or filter paper made of glass fibers.

4. The analysis chip according to claim 1, wherein said probe molecules are fixed to the carrier via a linker.

5. A process for preparing affinity reaction probe detection/analysis chips, comprising the steps of:
stacking chips each comprising a retainer ring that is labelled with individual identifying signals and which has a through-hole filled with or retaining a carrier;
passing a solution of preliminarily synthesized or naturally extracted probe molecules through said through-holes in the stacked chips; and
fixing said probe molecules to a surface of said carrier using a linker.

6. A process for preparing affinity reaction probe detection/analysis chips, comprising the steps of:
providing chips each comprising a retainer ring that is labelled with individual identifying signals and which has a through-hole filled with or retaining a carrier;
stacking the chips; and
synthesizing oligonucleotides as probe molecules on a surface of said carrier.

7. An affinity reaction probe detection/analysis chip assembly which is a stack of the affinity reaction probe detection/analysis chips according to claim 1, wherein the probe molecules fixed to the carrier are capable of causing different specific binding reactions, the through-holes in the individual chips being joined to form a channel through which an analyte can pass.

8. An apparatus for preparing affinity reaction probe detection/analysis chips, comprising:
a stack of chips each comprising a retainer ring that is labelled with individual identifying signals and which has a through-hole filled with or retaining a carrier; and
means for successively flowing reagents through a channel formed by joining the through-holes in said stacked chips.
